(19) United States
(12) Patent Application Publication
(10) Pub. No.: US 2022/0257277 A1
(43) Pub. Date: Aug. 18, 2022

(54) COLD CUTTING POLYPECTOMY SNARE

(71) Applicant: THE JOHNS HOPKINS UNIVERSITY, Baltimore, MD (US)

(72) Inventor: Anthony N Kalloo, Baltimore, MD (US)

(21) Appl. No.: 17/618,793

(22) PCT Filed: Jun. 12, 2020
(86) PCT No.: PCT/US2020/037544

§ 371 Date: Dec. 13, 2021

Related U.S. Application Data
(60) Provisional application No. 62/860,855, filed on Jun. 13, 2019.

(51) Int. Cl. A61B 17/3205; D07B 1/06; D07B 5/10

(52) U.S. Cl. CPC A61B 17/32056; A61B 2017/00269; D07B 5/10; D07B 1/068

(57) ABSTRACT

The present invention is directed to a cold cutting polypectomy snare. The cold cutting polypectomy snare has a cross section optimized for polypectomy. In some embodiments, the snare may have a uniform cross section along a length of the snare. In other embodiments, the snare may include a first side and a second side, wherein the first and second sides each have a unique cross section. Further, the cross-sections of the first side and the second side can be configured to couple together when a diameter of the snare is decreased to affect a cutting of a polyp.

### CROSS REFERENCE TO RELATED APPLICATIONS

This application claims the benefit of U.S. Provisional Patent Application No. 62/860,855 filed on Jun. 13, 2019, which is incorporated by reference, herein, in its entirety.

### FIELD OF THE INVENTION

The present invention relates generally to medical devices. More particularly, the present invention relates to a cold cutting polypectomy snare.

### BACKGROUND OF THE INVENTION

Polypectomy during colonoscopy is used to prevent cancer. It includes the removal of polyps of various sizes from the colon. While removing the polyps is a clear necessity, there are a number of different tools for effecting the removal of these polyps. These tools include hot forceps, hot snares, cold forceps, and cold snares. Cold tools eliminate the risk of perforation and delayed bleeding. Further, for polyps smaller than 5 mm cold snaring is more effective. While cold snaring is emerging as a preferred method for polypectomy, new tools for performing cold snare polypectomy are needed.

Accordingly, there is a need in the art for a device optimized to perform cold cutting polypectomies.

### SUMMARY OF THE INVENTION

The foregoing needs are met, to a great extent, by the present invention which provides a device for polypectomy including a wire forming a loop and the loop defining an inner diameter. The wire has a cross-section configured for cutting a polyp. The diameter of the loop can be decreased to affect a polypectomy.

In accordance with an aspect of the present invention, the wire is formed from a metal. The wire is formed from a shape-memory metal. The cross-section of the wire can have a sharp edge for cutting the polyp.

In accordance with another aspect of the present invention, a device for polypectomy includes a wire forming a loop and the loop defining an inner diameter. The loop has a first side and a second side. The wire has a first cross section on the first side and a second cross section on the second side. The diameter of the loop can be decreased to affect a polypectomy and wherein the first cross section is configured to couple with the second cross section to affect the polypectomy.

In accordance with still another aspect of the present invention, the wire is formed from a metal. The wire can be formed from a shape-memory metal. The cross-section of the wire includes a sharp edge. The sharp edge is formed by grinding and polishing. The device can also include a sheath.

In accordance with still another aspect of the present invention, a device for polypectomy includes a wire forming a loop and the loop defining an inner diameter. The wire includes a first portion having a cross-section configured for cutting a polyp and a second portion configured for bending.

In accordance with even another aspect of the present invention, the diameter of the loop can be decreased to affect a polypectomy. The wire is formed from a metal or is formed from a shape-memory metal. The second portion of the wire is formed from a wire cable. The first portion of the wire is formed from a shape memory metal. The first portion of the wire is formed from stainless steel. The first and second portions of the wire are coupled using one selected from a group consisting of welding and adhesive.

### BRIEF DESCRIPTION OF THE DRAWING

The accompanying drawings provide visual representations, which will be used to more fully describe the representative embodiments disclosed herein and can be used by those skilled in the art to better understand them and their inherent advantages. In these drawings, like reference numerals identify corresponding elements and:

FIG. 1A illustrates a top-down view of a polypectomy snare, according to an embodiment of the present invention.

FIG. 1B illustrates an exemplary cross-section of a wire forming a polypectomy snare, according to an embodiment of the present invention.

FIGS. 2A-2E illustrate exemplary cross-sections of a wire forming a polypectomy snare, according to an embodiment of the present invention.

FIG. 2F illustrates a polypectomy snare having the cross-sections illustrated in FIG. 2E.

FIGS. 3A and 3B illustrate polypectomy snares having sharpened portions disposed within a length of the snare, according to an embodiment of the present invention.

### DETAILED DESCRIPTION

The presently disclosed subject matter now will be described more fully hereinafter with reference to the accompanying Drawings, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

The present invention is directed to a cold cutting polypectomy snare. The cold cutting polypectomy snare has a cross section optimized for polypectomy. In some embodiments, the snare may have a uniform cross section along a length of the snare. In other embodiments, the snare may include a first side and a second side, wherein the first and second sides each have a unique cross section. Further, the cross-sections of the first side and the second side can be configured to couple together when a diameter of the snare is decreased to affect a cutting of a polyp. The snare of the present invention allows for a cleaner cut and a more precise specimen.

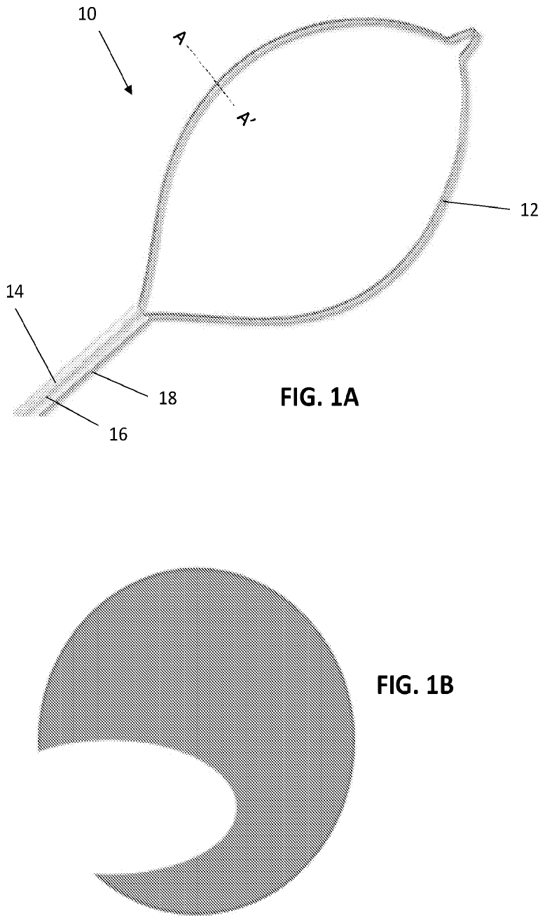

FIG. 1A illustrates a top-down view of a polypectomy snare, according to an embodiment of the present invention. As illustrated in FIG. 1A, the polypectomy snare 10 takes the form of a loop of wire 12. Ends 14, 16 of the loop of wire 12 wire come together and are housed in sheath 18. The ends 14, 16 of the loop of wire 12 wire are slidably disposed within the sheath 18, such that a diameter of the loop of wire 12 can be decreased to affect a polypectomy. The loop of wire 12 can be formed from a biocompatible metal or alloy, such as a shape memory metal alloy. The loop of wire 12 can also be formed from any other suitable material known to or conceivable to one of skill in the art. In some embodiments, the loop of wire 12 will take the form of a cable-style wire. In some embodiments the cable-style wire includes a hexagonal cross section. In some embodiments, the cable-style wire can be formed from steel. In other embodiments, a shape memory metal such as NiTiNol can be used. The diameter and the weight of the wire is chosen to allow for the wire to be pulled into and out of the sheath with a low amount of force.

FIG. 1B illustrates an exemplary cross-section of a wire forming a polypectomy snare, according to an embodiment of the present invention. The cross-section shown in FIG. 1B is taken along axis A-A′ of FIG. 1A. The cross-sectional shape of the wire is configured to optimize polypectomy. In the embodiment illustrated in FIG. 1B, the cross-section of the wire is uniform along a length of the wire. The cross-sectional shape of the wire can be formed by tooling or rolling the wire to achieve the shape.

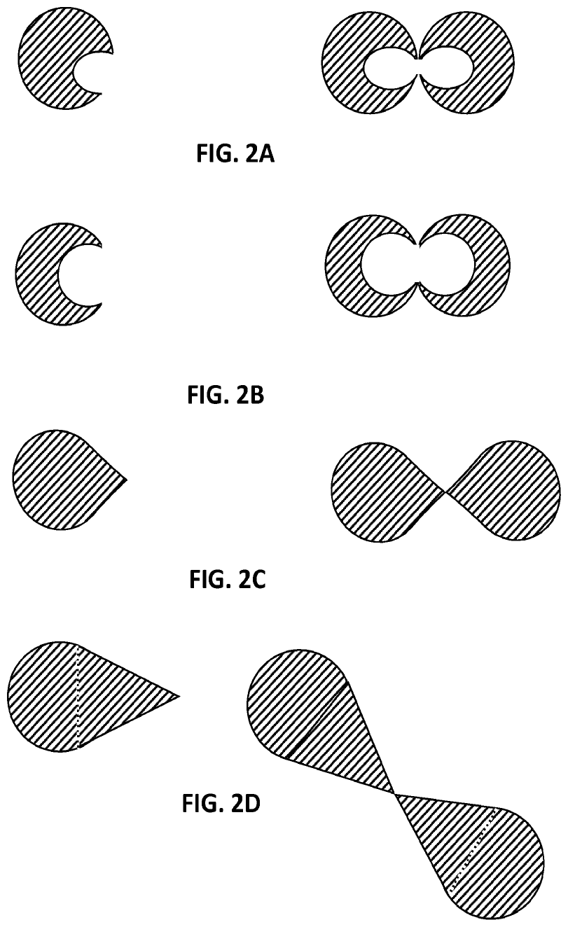

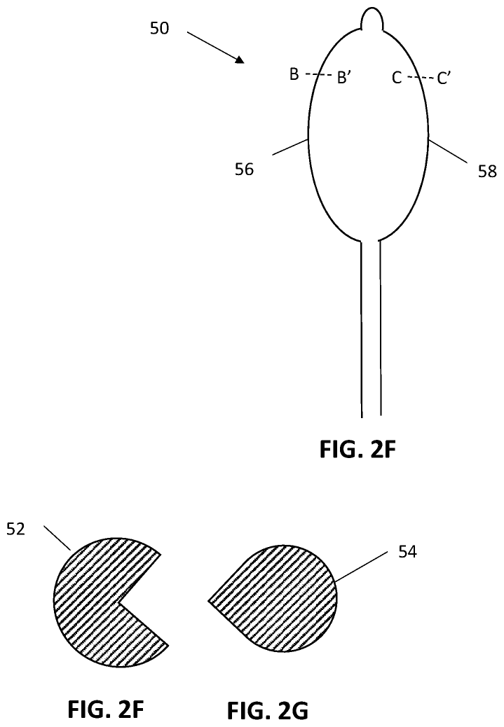

FIGS. 2A-2G illustrate exemplary cross-sections of a wire forming a polypectomy snare, according to an embodiment of the present invention. The cross-sectional shapes of the wires illustrated in FIGS. 2A-2G are configured to optimize polypectomy. In the embodiment illustrated in FIGS. 2A-2D the cross-section of the wire is uniform along a length of the wire. In other embodiments, it is possible that the cross-sectional shapes exemplified in FIGS. 2A-2G extend only along a portion of the wire loop. In the embodiment illustrated in FIG. 2E, the cross-section of the wire changes along the length of the wire. FIG. 2E illustrates a polypectomy snare 50 having the cross-sections 52, 54 illustrated in FIGS. 2F and 2G. A first half of the wire 56 has a first cross-section 52 taken along axis B-B′, as illustrated in FIG. 2F. A second half of the wire 58 has a second cross-section 54 taken along axis C-C′, as illustrated in FIG. 2G. In this exemplary embodiment, the first cross-section 52 and the second cross section 54 are configured to fit together in order to cut the polyp and perform the polypectomy. The sharp edge cuts the polyp. The wire can be formed from a biocompatible metal or metal alloy. The wire can also be formed from any other suitable material known to or conceivable to one of skill in the art. The cross-sectional shape of the wire can be formed by tooling or rolling the wire to achieve the shape. It should further be noted that while FIGS. 2A-2G illustrate a number of possible cross sectional shapes for the wire, any shape known to or conceivable to one of skill in the art could also be used.

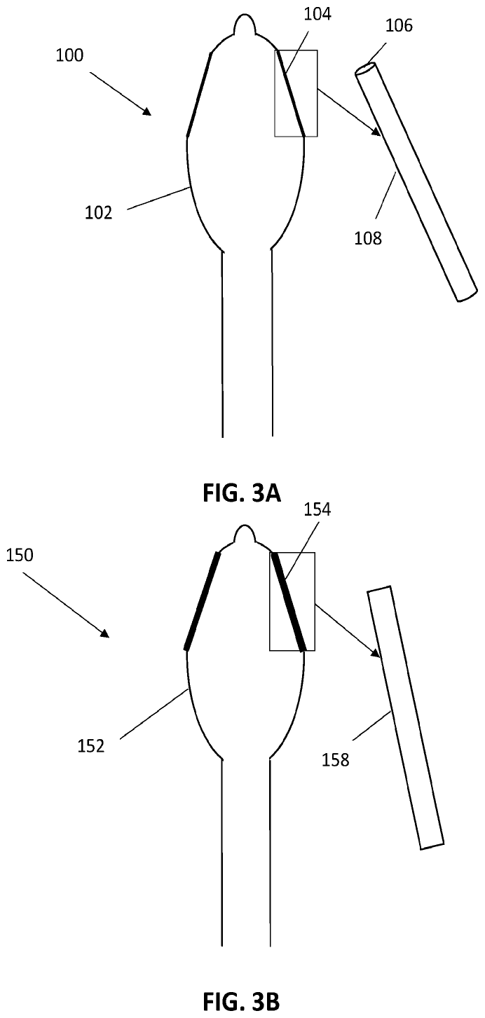

FIGS. 3A and 3B illustrate polypectomy snares having sharpened portions disposed within a length of the snare, according to an embodiment of the present invention. FIG. 3A illustrates an embodiment of a polypectomy snare 100 where a portion 104 of the wire loop 102 takes the form of a wire with a round cross section 106 and a sharpened edge 108. In the embodiment shown in FIG. 3B, a polypectomy snare 150 includes a portion 154 of the wire loop 152 that takes the form of a flat wire with a sharpened edge 158. The sharpened edge is achieved by grinding and polishing the portion to be sharpened. While FIGS. 3A and 3B show respective polypectomy snares 100 and 150 with two sharpened portions in each snare, any number and size of sharpened portion known to or conceivable to one of skill in the art could also be used. Further, while FIGS. 3A and 3B show snares with portions having round and flat cross sections, any cross sectional shape known to or conceivable to one of skill in the art could also be used. These differently shaped portions can be coupled to the wire loop in different ways, including but not limited to a laser weld directly to the wire loop, an adhesive to directly couple the portion to the wire loop, and an adhesive to couple the portion to a hypotube, which is in turn, laser welded to the wire. An exemplary adhesive is an adhesive cured with ultraviolet light, though any adhesive known to or conceivable to one of skill in the art could also be used.

With respect to FIG. 3A, in a preferred embodiment, the portion 104 is formed from a stainless steel round wire with a diameter of 0.015 inches. This stainless steel round wire is ground and polished to have a sharpened edge 108 with a width of 0.004 to 0.006 inches. This round wire is welded to a cable wire with a diameter of 0.016 inches at the distal tip and proximal snare section to allow for collapse and recovery of the snare hoop. This sub assembly is laser welded to a cable wire with a diameter of 0.036 inches to allow for advancement and retraction through a sheath. The sheath is preferably formed from a plastic such as high-density polyethylene (HDPE).

With respect to FIG. 3B, in a preferred embodiment, the portion 154 is formed from a flat wire having an exemplary dimension of 0.006 inches by 0.040 inches. The flat wire is laser welded to a cable wire with a diameter of 0.016 inches at the distal tip and proximal snare section to allow for collapse and recovery of the snare hoop. This sub assembly is laser welded to a cable wire with a diameter of 0.036 inches to allow for advancement and retraction through a sheath. The sheath is preferably formed from a plastic such as high-density polyethylene (HDPE).

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

1. A device for polypectomy comprising:
a wire forming a loop and the loop defining an inner diameter;
wherein the wire comprises a cross-section configured for cutting a polyp;
wherein the diameter of the loop can be decreased to remove the polyp and affect the polypectomy.

2. The device for polypectomy of claim 1 wherein the wire is formed from a metal.

3. The device for polypectomy of claim 1 wherein the wire is formed from a shape-memory metal.

4. The device for polypectomy of claim 1 wherein the cross-section comprises a sharp edge.

5. The device for polypectomy of claim 1 further comprising a sheath.

6. A device for polypectomy comprising:
a wire forming a loop, the loop defining an inner diameter, and the loop having a first side and a second side;
wherein the wire comprises a first cross section on the first side and a second cross section on the second side;
wherein the inner diameter of the loop can be decreased to affect the polypectomy and wherein the first cross section is configured to couple with the second cross section to affect the polypectomy.

7. The device for polypectomy of claim 6 wherein the wire is formed from a metal.

8. The device for polypectomy of claim 6 wherein the wire is formed from a shape-memory metal.

9. The device for polypectomy of claim 6 wherein one of the first or second cross-sections comprises a sharp edge.

10. The device for polypectomy of claim 6 further comprising a sheath.

11. A device for polypectomy comprising:
a wire forming a loop and the loop defining an inner diameter;
wherein the wire comprises a first portion comprising a cross-section configured for cutting a polyp and a second portion configured for bending;
wherein the inner diameter of the loop can be decreased to affect a polypectomy.

12. The device for polypectomy of claim 11 wherein the wire is formed from a metal.

13. The device for polypectomy of claim 11 wherein the wire is formed from a shape-memory metal.

14. The device for polypectomy of claim 11 wherein the cross-section comprises a sharp edge.

15. The device of claim 14 wherein the sharp edge is formed by grinding and polishing.

16. The device for polypectomy of claim 11 further comprising a sheath.

17. The device for polypectomy of claim 11 wherein the second portion of the wire is formed from a wire cable.

18. The device for polypectomy of claim 11 wherein the first portion of the wire is formed from a shape memory metal.

19. The device for polypectomy of claim 11 wherein the first portion of the wire is formed from stainless steel.

20. The device for polypectomy of claim 11 wherein the first and second portions of the wire are coupled using one selected from a group consisting of welding and adhesive.
